(19) United States
(12) Patent Application Publication
(10) Pub. No.: US 2011/0282198 A1
(43) Pub. Date: Nov. 17, 2011

(54) RAPID RESPONSE ANGIOGRAPHIC CATHETER

(75) Inventor: Allen J. Tower, North Lawrence, NY (US)

(73) Assignee: Numed, Inc., Nicholville, NY (US)

(21) Appl. No.: 12/781,420

(22) Filed: May 17, 2010

(51) Int. Cl. A61B 6/00; A61M 25/10

(52) U.S. Cl. 600/435

(57) ABSTRACT

An angiographic catheter having an elongated flexible insertion tube having a cylindrical inflatable sheath circumferentially mounted thereon. The sheath contains a series of discharge ports at its distal end. A fluid is introduced into the proximal end of the sheath under sufficient pressure so that the sheath is fully inflated and fluid is discharged into a desired target area within the patients body.

### FIELD OF THE INVENTION

This invention relates generally to angiographic catheters and more specifically to an angiographic catheter that has a rapid rate of delivery.

### BACKGROUND OF THE INVENTION

The increasing complexity of diagnostic catheterization, particularly cardiac catheterizations in patients with congenital heart disease or the like, have lead to the development of a wide variety of catheters. Among these are angiographic catheters that attempt to solve a number of problems found in children and older adults. These catheters typically involve an insertion tube having discharge ports located at its distal end section. A radiographic fluid is delivered to the ports by an independent lumen that is generally housed inside the insertion tube. The acceptable outside diameter of the catheter insertion tube for a particular procedure usually dictates the size of the delivery lumen and thus the rate at which the radiographic fluid can be supplied to the discharge ports. Often times these flow rates are relatively slow and can add to the potential risk to the patient. In addition, these insertion tubes are relatively rigid member that tend to become unstable during insertion due to the forces exerted thereon by the fluid flowing through the system during the catheterization procedure.

### SUMMARY OF THE INVENTION

It is therefore a primary object of the present invention to improve angiographic catheters.

A further object of the present invention is to increase the flow rate of radiopaque fluid through an angiographic catheter while at the same time decreasing the cross sectional area of catheter's insertion tube.

A still further object of the present invention is to reduce the procedure time required to complete an angiographic catheterization.

Another object of the present invention is to reduce a patient's risk during an angiographic catheterization procedure.

These and other objects of the present invention are attained by means of an angiographic catheter having a narrow diameter insertion tube for slidably containing a guide wire. A thin wall cylindrical inflatable sheath is circumferentially mounted upon the insertion tube and extends along the tube between its distal and proximal ends. A series of discharge ports are formed in the side all of the sheath at its distal end. A pump is connected into the proximal end of the sheath for delivering a radiographic fluid into the sheath to fully inflate the sheath and to cause the fluid to be discharged at a rapid rate through the discharge ports.

### BRIEF DESCRIPTION OF THE DRAWINGS

For a better understanding of these and other objects of the invention, reference will be made to the following detailed description of the invention which is to be read in association with the accompanying drawings wherein:

FIG. 1 is a side elevation of the present angiographic catheter showing the outer sheath in a deflated condition;

FIG. 2 is also a side elevation of the present angiographic catheter showing the outer sheath in an inflated condition; and

FIG. 3 is a sectional view taken along lines 3-3 in FIG. 2.

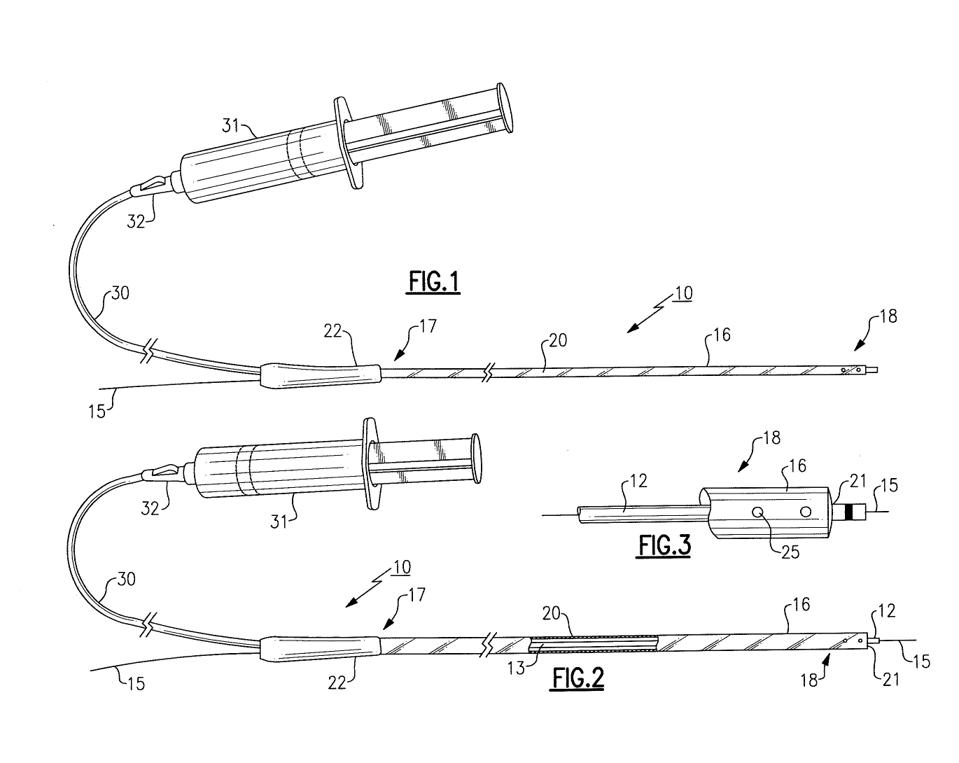

### DETAILED DESCRIPTION OF THE INVENTION

Turning now to the drawings, there is illustrated an angiographic catheter, generally referenced 10, that embodies the teaching of the present invention. The catheter includes an insertion shaft 12 that has a central lumen 13 contained therein for slidably supporting a guide wire 15 therein. The insertion shaft is a flexible member, fabricated of a polyamide material that has sufficient beam strength so that the shaft can be percutaneously inserted into a desired body cavity.

A cylindrical sheath 16 is circumferentially mounted upon the insertion tube which axially spans between the proximal end section 17 of the tube to the distal end section 18 of the tube. The sheath is fabricated of an inflatable material 20 having an inside diameter that is larger than the outside diameter of the insertion tube. The two opposed ends of the sheath are heat sealed to the outside of the insertion tube at sections 21 and 22 to provide a fluid tight joint between the two members. Preferably the sheath is fabricated of a nylon having a wall thickness of about 0.0005 inches One or more discharge ports 25 are passed through the side wall of the sheath at its distal end.

An extension tube 30 is passed into the proximal end of the sheath and is snap-fitted to a syringe 31 via a connector 32. The syringe contains a radiographic fluid and is arranged to deliver the fluid under sufficient pressure to fully inflate the sheath and to dispense the fluid rapidly through the one or more discharge ports.

At the start of an angiographic procedure, the sheath is deflated into a compact package about the insertion tube so that the entire insertion assembly has a diameter that is slightly larger than that of the insertion tube. Accordingly the present device can pass through small channels and transcend tight bends with relative ease. Once positioned within the desired target region the sheath is fully inflated to establish a relatively large annular shaped flow passage around the sheath that provides for a high rate of fluid flow through the catheter to the discharge port or ports. As the inflation fluid moves along the inflation passage it must work to inflate the sheath and hold it inflated. The fluid thus acquires more and more energy as it approaches the discharge port or ports. As a result, the fluid is rapidly ejected through the port or ports at a speed that is considerably higher than that attainable by more conventional devices presently found in the prior art. Typically during the inflation period, the inflated sheath will come in contact with the patient's tissue at various points along the percutaneous passage and thus stabilize the insertion tube assembly during the angiographic procedure.

When the procedure is completed the sheath is deflated with the help of the syringe and the insertion tube assembly is withdrawn from the patient.

As should now be evident, the present angiographic catheter, while simply in designs affords a number of advantages over those in present day usage. The use of a collapsible outer shaft or sheath provides for ease of entry and removable of the insertion assembly into and out of typically hard to reach body cavities. The annular flow passage that is established by the inflated sheath provides for an extremely high rate of flow into the target area thus allowing the procedure to be more rapidly completed which, in turn, poses less risk to the patient. Lastly, when inflated the insertion tube assembly becomes well anchored within the percutaneous passage thus resulting in a more stable delivery platform again resulting a shorter procedure period.

While the invention has been described with reference to preferred embodiments, it will be understood by those skilled in the art that various changes may be made and equivalents may be substituted for elements thereof to adapt to particular situations without departing from the scope of the invention. Therefore, it is intended that the invention not be limited to the particular embodiments disclosed as the best mode contemplated for carrying out this invention, but that the invention will include all embodiments falling within the scope and spirit of the appended claims.

1. A rapid response angiographic catheter for introducing a fluid into a body cavity that includes:
a flexible elongated insertion tube having a distal end section, a proximal end section and an axially extended lumen for slidably containing a guide wire;
a cylindrical inflatable sheath circumferentially mounted upon said insertion tube that extends between the proximal and distal end sections of said insertion tube;
said sheath having one or more discharge ports located at its distal end; and
means for delivering a flow of fluid under pressure into the proximal end of said sheath for fully inflating said sheath and discharging said fluid through said one or more discharge ports into a target area.

2. The catheter of claim 1, wherein said means for delivering said fluid into said sheath is a syringe.

3. The catheter of claim 2, wherein said sheath is fabricated of a thin wall nylon.

4. The catheter of claim 3, wherein said sheath has a wall thickness of about 0.0005 inches.

5. The catheter of claim 2, wherein said fluid is radiopaque.

6. A method of introducing a fluid into a target area of a body that includes the following steps:
mounting an inflatable sheath circumferentially upon an elongated insertion tube so that said sheath covers substantially the entire length of said tube between its proximal end and its distal end;
providing one or more discharge ports at the distal end of said sheath;
positioning said insertion tube with said sheath in an deflated condition within a bodily target area; and
introducing a fluid into said sheath under sufficient pressure to fully inflate said sheath and discharge said fluid through said one or more discharge ports.

7. The method of claim 6, wherein the sheath is fabricated of nylon.

8. The method of claim 7, that includes the further step of heat sealing said sheath to said insertion tube.

9. The method of claim 6, wherein said fluid is introduced into said sheath through the proximal end of said sheath.

10. The method of claim 6, wherein said fluid is radiopaque.
